(19) United States
(12) Patent Application Publication
(10) Pub. No.: US 2025/0107836 A1
(43) Pub. Date: Apr. 3, 2025

(54) GAS DOSING UNIT FOR GAS SUPPLY TO A CRYOINSTRUMENT

(71) Applicant: Erbe Elektromedizin GmbH, Tuebingen (DE)

(72) Inventors: Jürgen HILLER, Dettingen (DE); Kai Guendisch, Reutlingen (DE)

(21) Appl. No.: 18/894,459

(22) Filed: Sep. 24, 2024

(30) Foreign Application Priority Data
Sep. 28, 2023 (EP) 23200465.5

(51) Int. Cl. A61B 18/02; A61B 18/00

(52) U.S. Cl. CPC A61B 18/02; A61B 2018/00642; A61B 2018/00672; A61B 2018/00678; A61B 2018/00744

(57) ABSTRACT

A gas dosing unit is configured to adjust a desired gas flow and includes a pressure control module having a pressure reduction device. In case of normal room temperature, a control device controls the pressure reduction device for adjusting a desired pressure or a desired gas stream. If the room temperature and/or the temperature of the connected gas storage is lower and accordingly, the gas pressure applied at the inlet connection of the gas dosing unit is insufficient for the correct operation of the pressure control module, the control device activates a compressor arranged in the gas path, which increases the pressure upstream of the pressure control module to a value appropriate for the correct operation of the pressure control module. In doing so, the cryoinstrument can be correctly activated and used independent of the room temperature and the pressure inside the pressure storage. Operating limitations existing otherwise are avoided.

### CROSS REFERENCE TO RELATED APPLICATION

This application claims the benefit of European Patent Application No. 23200465.5, filed Sep. 28, 2023, which is incorporated herein by reference in its entirety.

### TECHNICAL FIELD

The invention refers to a gas dosing unit, which is configured for gas supply of a cryoinstrument, as well as also a combination of a gas dosing unit, gas storage and cryoinstrument. Such cryoinstruments can be used, for example, for biopsy or also for other medical purposes, for example, cryoablation.

### BACKGROUND

The gas dosing unit, according to the invention, serves for supply of the cryoinstrument with a gaseous coolant, such as particularly CO₂, N₂O or another gas appropriate for cold production, particularly and at least triatomic gas. The cryoinstrument operates according to the cryoprinciple, according to which an instrument tip being in contact with biological tissue is cooled down so that tissue freezes and sticks to the instrument tip. In this manner, the tissue sticking to the instrument tip can be extracted from the patient by means of the instrument, for example, for further examination or also for other purposes.

For supply of a cryoablation device US 2002/0068929 A1 discloses a gas supply device having a compression vessel that comprises a flexible wall, which divides the interior of the compression vessel into two compartments. Due to pressure application of one of the compartments, the medium present in the other compartment can be pressurized and can be supplied to a gas consumer.

For supply of cryoinstruments with compressed gas and for cooling their tips by means of the Joule-Thompson-effect WO 2004/064914 A2 discloses a gas dosing unit in which gas, which flows back from instruments under low pressure, is compressed by means of a compressor and then again supplied with high pressure to the instruments via so-called flow regulators.

In addition, EP 3 854 334 A1 discloses a gas dosing unit for gas supply of a cryoinstrument. The dosing unit comprises an inlet connection for a gas storage and an outlet connection for the instrument. A pressure control module is arranged between the inlet connection and the outlet connection. This comprises a pump, which can be configured as double-acting piston pump, for example, and which is configured to feed gas from the pressure storage into a buffer vessel. Assigned to the buffer vessel is a tempering device that can serve for heating or also cooling of the buffer vessel. In addition, the pressure control module comprises a pressure control valve in order to supply gaseous coolant from the buffer vessel to the outlet connection and thus to the instrument in a specific and controlled manner. A control device is thereby configured to detect the pressure and/or the temperature inside the buffer vessel and to control its tempering.

### SUMMARY

When using cryoinstruments, particularly during biopsy, frequently a very quick activation of the probe is desired in order to keep the time short that is required for the biopsy. For example, a usual application of a biopsy probe takes only 3 to 5 s, so that the freezing power at the instrument tip after the activation of the gas dosing unit shall be provided immediately, as far as possible.

Gas shall mainly serve as coolant for operating the cryoinstrument that shows a strong Joule-Thomson-effect and thus a distinct cold development when expanded. Such a gas is CO₂ or also N₂O. Also other triatomic or polyatomic gases, particularly gases having polar molecules, work well.

Typically, such gases are taken from pressure storages, for example gas bottles or the like, in which the gas is provided in liquefied form due to pressurization. Typically, this gas is supplied to the cryoinstrument via a pressure control module, whereby it has shown that the freezing power of the cryoinstrument can start delayed after activation if the temperature of the gas storage, for example due to a low room temperature or for other reasons, drops below a temperature limit. For example, at room temperatures below approximately 18° C., the freezing power of the cryoinstrument can start delayed or can remain below the required power. Finally, this can extend the required application duration or can make the application impossible, which is declined by treating persons. Particularly, remarkably long and thin instruments are concerned.

Therefrom one object is derived on which the invention is made, to provide a gas dosing unit with which the cryoinstrument operates after activation without delay and with the desired cooling power, also in case of low temperatures of the gas storage.

This object is solved by means of a gas dosing unit as described herein.

The gas dosing unit, according to the invention, comprises a pressure control module that is arranged between an inlet connection and an outlet connection. A gas storage can be connected to the inlet connection, while a cryoinstrument can be connected to the outlet connection. The pressure control module is configured to guide gas from the inlet connection to the outlet connection, whereby the gas thereby flows through the pressure reduction device. At least one pressure sensor is arranged upstream the pressure reduction device in order to measure the gas pressure of the gas supplied to the pressure reduction device and to compare it with a limit value Pmin. The control device is configured to activate the compressor in order to increase the pressure of the gas supplied to the pressure reduction device to or above the limit value Pmin if the pressure of the gas coming from the gas storage is below the limit value Pmin.

The compressor is connected to a motor and is driven by the latter. The motor is connected with the control device that operates the motor with a variable rotational speed that is set by the control device. Thereby, the control device, which is concurrently connected with at least one pressure sensor, is configured to build up a sufficiently high pressure by means of the compressor, so that the pressure reduction device provides a controlled gas flow with the desired amount to the cryoinstrument.

In addition, the control device can be configured to drive—that means activate—the motor and together therewith the compressor only if an activation switch is activated, which is arranged on the instrument or separate therefrom and which is connected with the control device. In doing so, it can be guaranteed that the compressor runs only for activating the instrument, but not during idle times. Thereby, it is possible to omit buffer volumes between the compressor and the downstream valve (activation valve) and also to minimize the line volume that is present there. Thereby, on the other hand, a quicker pressure build-up can be achieved in case of activation of the compressor. This also contributes to noise reduction and thus to quiet at the patient. In addition, exemplars of the compressor that operate particularly smooth are preferred, which then have at least two or also multiple pistons. Slowly rotating compressors with maximum rotational speeds below 300 rpm have thereby shown to run particularly smooth, which have an approximately square bore-to-stroke-ratio and the connection rod of which is at least 5 times, better 7 to 8 times as long as the crank radius of its crankshaft. The crank radius is identical to the eccentricity of the crank pin of its crankshaft.

In a preferred embodiment, the cryoinstrument comprises a gas feedback line connected to the gas dosing unit. The gas dosing unit comprises a gas path for the gas that was fed back in which a mass flow sensor is arranged. The latter is preferably connected with the pressure control module in order to adjust the pressure of the gas fed back to the cryoinstrument to a value that fits a desired mass flow value. The control device of the pressure control module is configured to compare the desired mass flow with the real, measured mass flow and to increase or decrease the pressure of the gas supplied to the instrument in order to establish the desired equality of the desired mass flow and the measured mass flow. Particularly, in case of very thin and/or very long instruments, the pressure required for this purpose can be remarkably higher than the pressure provided by the gas storage. In this case, the control device is configured to activate the compressor.

In a possible other embodiment, the pressure control module comprises a first pressure sensor between the inlet connection and the compressor and a second pressure sensor between the compressor and the pressure reduction device. Both pressure sensors are preferably connected with the control device. Moreover, the pressure reduction device can have a controllable valve, the actuator of which is connected with the control device.

A flow measurement device can be arranged between the valve and the outlet connection or, as mentioned above, in a gas feedback line. For example, the flow measurement device can comprise a throttle as well as pressure sensors arranged upstream and downstream of the throttle. Other configurations with impeller, ultrasound or heat measurement are possible. For example, one of the pressure sensors can be arranged between the valve and the throttle, while the other pressure sensor can be arranged between the throttle and the outlet connection. Both pressure sensors can be connected with the control device. The latter can be configured to adjust the valve so that a desired pressure drop is present at the throttle and thus a desired mass flow in order to supply the cryoinstrument.

The valve controlled by the control device can have a closed position and an open position as well as, preferably, intermediate control positions in which it throttles the gas stream flowing therethrough to a greater or lesser extent. Thereby, the valve is configured to keep the mass flow of the gas toward the instrument constant and to thereby as well eliminate pressure pulsations that result or can result from the operation of the compressor.

The control device can take different modes of operation. After activation, due to actuation of an activation switch, the control device can first check whether the pressure upstream of the valve exceeds the minimum pressure. This pressure can be detected by means of the first pressure sensor or, potentially, also with the second pressure sensor. The control device can be configured to operate the motor of the compressor first with the preset, preferably, the highest possible rotational speed if the pressure is below the minimum pressure Pmin. The control device can be configured to be in this mode of operation for a defined time phase, for example, 1 or 2 s. It is, however, also possible to terminate this operating mode as soon as the pressure monitored by means of the second pressure sensor in the line between the compressor and the pressure reduction device has reached a maximum pressure Pmax, in order to then change to the second operating mode. While the rotational speed of the motor is maintained on a specific value in the first operating mode, the control device can be configured to control the rotational speed of the motor in the second operating mode according to the desired pressure in the line between the compressor and the pressure reduction device. In doing so, a sufficient pressure is always provided to the pressure reduction device in order to supply the cryoinstrument with the required mass flow of gaseous cryofluid.

The control device can be configured to operate in a third operating mode in which the motor is not driven and the compressor stands still. The control device can use the pressure detected by the first and/or the second pressure sensor for this purpose. If the pressure of the gas supplied by the gas storage is between the minimum pressure Pmin and the maximum pressure Pmax, a sufficient output pressure for the correct operation of the pressure reduction device is provided. The gas stream can then flow through the working spaces of the non-operating compressor (the pistons of which then rest immovably) or through a bypass deblocked for this purpose to the valve by means of which the control device then controls the desired gas stream.

### BRIEF DESCRIPTION OF THE DRAWINGS

Further details of embodiments of the invention are subject of the drawing, as well as the associated description or of dependent claims.

In the drawing embodiments of the invention are illustrated. The drawing shows:

FIG. 1 a gas dosing unit with a cryoinstrument connected thereto in a schematic, total illustration,

FIG. 2 the gas dosing unit in a schematic illustration,

FIG. 3 a diagram for illustration of the operation of the components of the gas dosing unit,

FIG. 4 the compressor and parts of the pressure reduction device in a schematic illustration,

FIGS. 5 to 8 alternative configurations of the gas dosing unit according to the invention.

### DETAILED DESCRIPTION

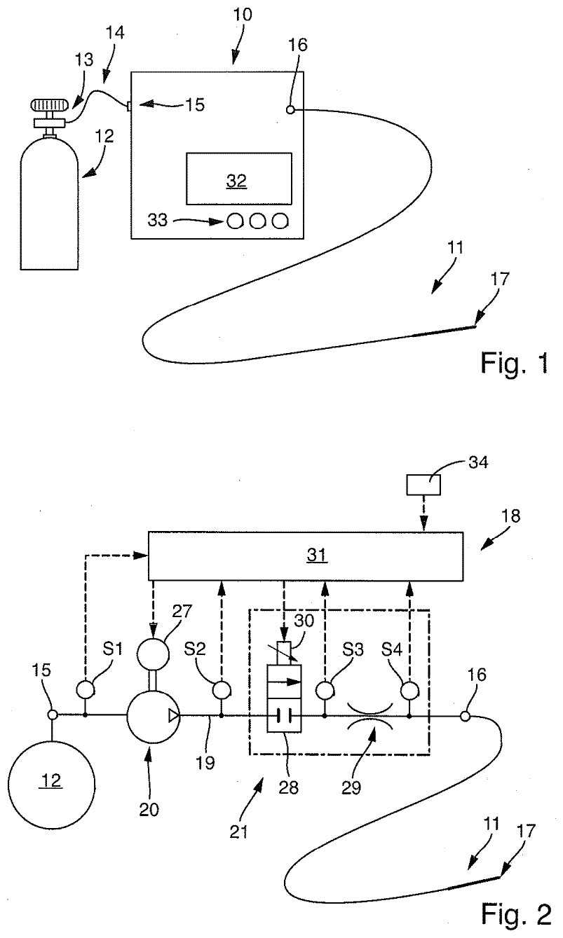

In FIG. 1 a gas dosing unit 10 is illustrated that serves to supply a cryoinstrument 11 with gas from a gas storage 12. The gas storage 12 can be a gas bottle, for example, in which the gas serving for supply of the instrument 11 is provided in a liquefied manner under pressure. Typical gases for this purpose are CO₂, N₂O or other polyatomic gases having a boiling temperature remarkably below 0° C. The pressure present in the gas storage 12 is typically between 40 and 60 bar.

The gas storage 12 comprises a connection 13 from which a line 14 leads to an inlet connection 15 of the gas dosing unit 10, preferably without intermediate connection of a pressure reduction valve. The gas dosing unit 10 comprises, in addition, an outlet connection 16 to which the cryoinstrument 11 is connected and via which it is supplied with gas for cryocooling of its tip 17.

Particularly, the cryoinstrument 11 is a long, slim, flexible instrument having a probe hose that is closed at the tip 17 by means of an end piece typically consisting of metal. In this end piece a capillary opens out from which the gas supplied by the gas dosing unit 10 exits and thereby expands and thus cools the tip 17 of cryoinstrument 11 from the inside by means of the Joule-Thomson-effect. Thereby, temperatures remarkably below 0° C. are reached, so that tissue can freeze to and stick strongly to the tip 17.

The outer diameter of cryoinstrument 11 can be less than 2 mm. Accordingly narrow are the lumen provided therein and the capillary arranged inside the lumen. The length of the cryoinstrument 11 can exceed 1 m remarkably. Also, outer diameters below 1 mm and/or lengths exceeding 2 m are possible, wherein the gas pressure of the supplied gas has to be sufficiently high in order to overcome the resulting flow resistances. The cryoinstrument having a length of at least 1.5 m, 2 m or up to or exceeding 2.50 m is suitable, particularly if it has an outer diameter of less than 2 mm or less than 1 mm, for extracting a tissue sample (biopsy) in the bile duct of a patient. The invention extends also to carrying out such a biopsy with a cryoinstrument 11 and a gas dosing unit 10 of the described configuration. Such a cryoinstrument 11 can be particularly advantageously operated with a gas dosing unit 10 of the described configuration, which supplies CO₂ (or another suitable gas) with sufficient pressure that can exceed the pressure of the gas storage 12. The gas dosing unit 10 is particularly suitable to overcome the increased inner flow resistances resulting from the length and the low diameter of the cryoinstrument 11 and to achieve a cooling effect at its tip 17. Such slim cryoinstruments 11 can also be used for biopsy at other locations of the body of the patient in other narrow body lumina.

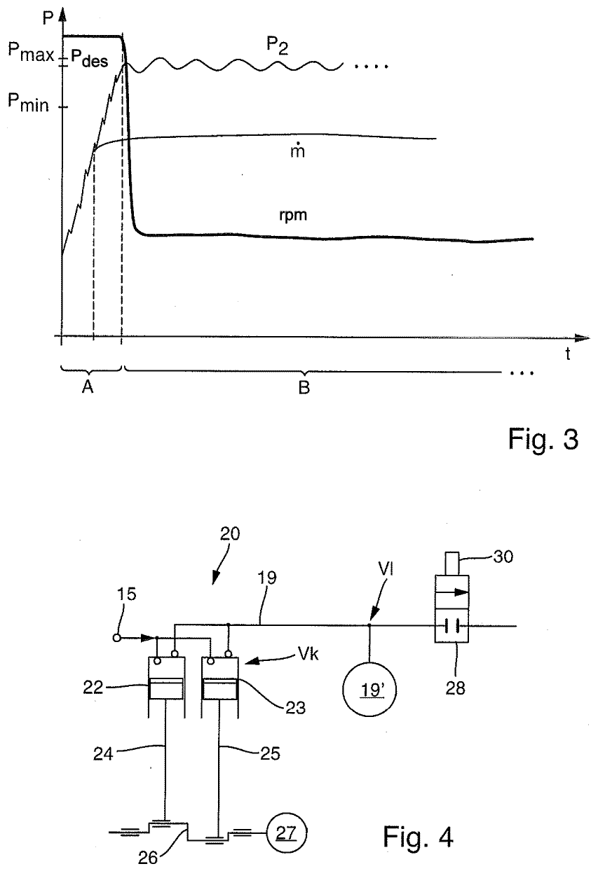

For reliable gas supply of cryoinstrument 11, the gas dosing unit 10 is provided, the basic configuration of which is apparent from FIG. 2. It comprises a pressure control module 18, illustrated in FIG. 2, in which a gas path 19 is formed, extending from the inlet connection 15 to the outlet connection 16, whereby gas passes through the gas path from the inlet connection 15 to the outlet connection 16. The gas path 19 comprises a compressor 20 and—in flow direction after the latter—a pressure reduction device 21.

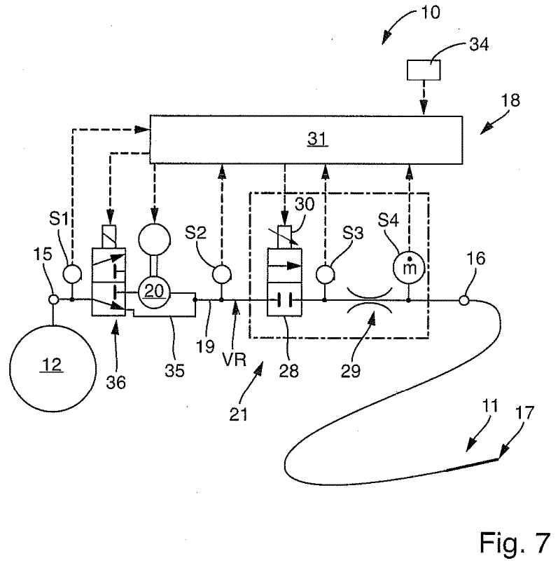

The compressor is preferably a piston compressor, further preferably a multi-piston compressor, as schematically illustrated in FIG. 4. Its two cylinders 22, 23 contain pistons that are connected with crank pins of a crankshaft 26 via connection rods 24, 25, wherein the crankshaft can be rotatingly driven with variable rotational speed by a motor 27. The eccentricity of the crank pins of crankshaft 26 is thereby preferably small compared to the length of the connection rods 24, 25, that means preferably less than a fifth or an eighth of their length. However, it has to be noted that instead of piston compressors, also other compressors can be used, for example rotary displacement pumps, gear pumps or rotary piston pumps or the like.

The two pistons limit the displacement volume that can be in total between one and three cubic centimeters. The adjoining volume V1 of gas path 19 is individually illustrated in FIG. 4 by means of the pressure storage symbol 19′. The volume V1 is at most 5, preferably at most 3 to 4 times larger than the displacement volume of compressor 20. In doing so, it is guaranteed that the volume V1 is filled with a few rotations of the crankshaft 16 and has the required pressure.

As apparent from FIG. 2, multiple pressure sensors S1 to S4 are arranged in the gas path 19. A first pressure sensor S1 is arranged between the inlet connection 15 and the compressor 20 and provides a signal that characterizes a first pressure P1. A second pressure sensor S2 is arranged between the compressor 20 and the pressure reduction device 21 and provides a signal that characterizes a second pressure P2. A third pressure sensor S3 can be arranged in the pressure reduction device between a valve 28 arranged therein and a downstream throttle 29 and provides a signal that characterizes a third pressure P3. For determination of the mass flow, a throttle 29 can be arranged behind and a fourth pressure sensor S4 can be arranged between the throttle 29 and the outlet connection 16. The fourth pressure sensor S4 provides a signal that characterizes a fourth pressure P4. The pressure difference P3-P4 characterizes the mass flow m. The valve 28, the throttle 29 and the two pressure sensors S3, S4 are in this embodiment part of the pressure reduction device.

The valve 28 can be configured as 2-port/2-way-valve and can be provided with an actuator 30 in order to take different throttle positions between the closed position and the open position. It serves as activation valve in order to deblock or stop the gas flow.

The pressure sensors S1 to S4 are connected to a control device 31 that is, in addition, configured to control the motor 27 and the actuator 30. The control device 31 can be a computer, a microcontroller or another digital or analog control circuit consisting of one or more units. In addition, it can be connected to an indication device 32 (FIG. 1) and one or more input means 33 and can be arranged on a housing of the gas dosing unit. Via the indication device 32 and the input means 33 operating conditions can be indicated and preset.

Moreover, the control device 31 is connected with an activation switch 34 (FIG. 2), that can be arranged on the housing of the dosing unit 10, as individual switch, for example as foot switch, or that can also be arranged on the instrument. The latter particularly if the instrument is provided as handheld instrument for the open surgical use. In a typical use cryoinstrument 11 is, however, provided and configured as slim, flexible probe for the endoscopic use.

The gas dosing unit 10 described so far operates in cooperation with the cryoinstrument 11 and the gas storage 12 as follows:

For setup of the gas dosing unit 10, at first gas storage 12 is connected to the inlet connection 15, for example by means of line 14, and the gas dosing unit 10 is transferred into operational readiness. The gas dosing unit can thereby be configured as individual apparatus or as part of a comprising larger apparatus.

After connection of the gas storage 12 to the gas dosing unit 10, gas flows out of the gas storage 12 into the gas path 19 up to the closed valve 28 until the pressure P1 detected by gas sensor S1 corresponds substantially to the interior pressure of the gas storage 12. In addition, gas can also reach the valve 28 via check valves of compressor 20 that are schematically indicated by small circles in FIG. 4, so that also the pressure sensor S2 detects the respective gas pressure P2 and outputs a respective signal to the control device 31. The gas dosing unit 10 is now ready for operation.

For biopsy or another treatment, cryoinstrument 11 is connected to the outlet connection 16 and guided to the operation site with its tip 17. By actuating the activation switch 34, cryoinstrument 11 is activated, that means it starts to cool. In detail, the control device 31 first checks the pressure P2 at pressure sensor S2 after receiving the activation signal from the activation switch 34. If this pressure is higher than the minimum pressure Pmin required for a correct operation of the pressure reduction device 21 and the cryoinstrument 11 the compressor 20 remains deactivated, that is the control device 31 causes motor 27 not to rotate. It also outputs a signal to the actuator 30 in order to open valve 28. Gas now flows through throttle 29 toward the cryoinstrument 11 (and therefrom via a not further illustrated outlet back or into the environment). Thereby the pressure sensors S3 and S4 supply signals characterizing the respective pressure P3, P4 from which the control device 31 determines the differential pressure P3-P4 over throttle 29. In order to guarantee a specific gas stream (liter per minute) to the cryoinstrument, control device 31 influences actuator 30, so that it gradually further opens or closes valve 28. The control device 31 operates in connection with the pressure sensors S3 and S4 as well as actuator 30 as closed-loop PID control with, for example, compressor 20 standing still.

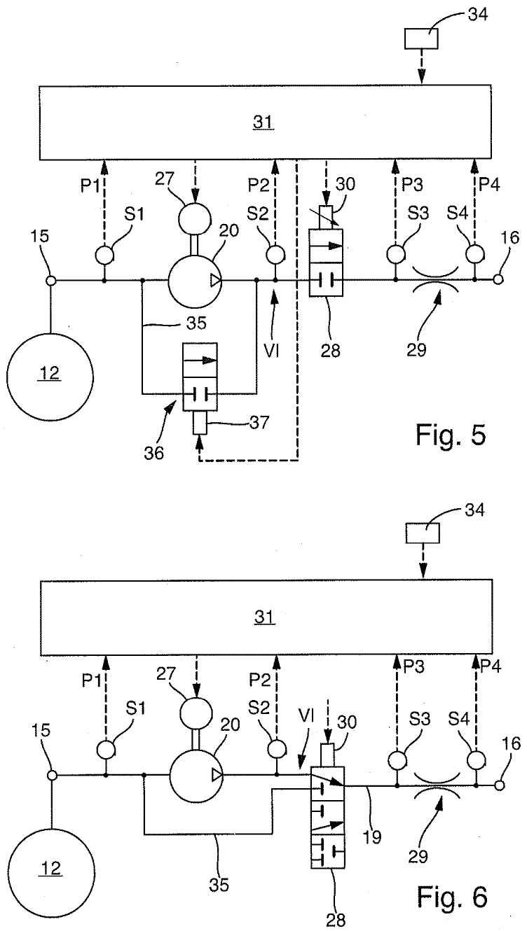

Another condition of operation is present if the temperature in the operation room or particularly the temperature of gas storage 12 is so low that the gas pressure P1 and/or P2 at sensor S1 and/or S2 drops below the minimum pressure Pmin. This condition of operation is illustrated in FIG. 3. Directly after initiating start of operation by actuating the activation switch 34, the control device 31 determines by checking the pressure P1 and/or P2 at sensor S1 and/or S2 that the present gas pressure is below the minimum pressure Pmin. The control device 31 is configured to thereupon operate motor 27 with maximum rotational speed. In this operating phase, control device 31 operates not as closed-loop PID control as otherwise, but it only monitors pressure P2 at the sensor S2 without thereby reducing the motor rotational speed. However, as soon as the pressure at the sensor S2 reaches the maximum pressure Pmax or also a pressure slightly below, for example, the pressure Pdes sufficient for operation of the pressure control, control device 31 terminates operation phase A and transitions into feedback control of pressure P2 according to the desired pressure Pdes. Thereby the control device 31 reduces the motor rotational speed and controls the latter operating as closed-loop PID control.

Due to the relatively small volume V1 enclosed between compressor 20 and valve 28 compared to the displacement volume of the compressor, the pressure variations around the desired value Pdes are still relatively high, as also shown in FIG. 3. The control device 31 is, however, configured to adjust these pressure variations by means of actuator 30 in a second closed control loop. The control device 31 thus forms a first closed control loop for the pressure by means of which the compressor rotational speed is controlled. It also forms a second closed control loop for the valve 28 for balancing the occurring pressure variations. The time constant of the second closed control loop is preferably remarkably shorter than the time constant of the first closed-control loop. It thereby results a smooth pressure progress without fluctuations, which are created by compressor 20, downstream valve 28 at pressure sensor S3. Accordingly, a constant gas mass flow m results, that means a constant gas flow. Also, the rotational speed rpm of motor 27 and thus compressor 20 remains relatively constant.

In the combination of operating phases A and B, the instrument 11 is provided with a gas stream suitable for the application practically immediately after activation of the cryoinstrument 11, that means after actuation of the activation switch 34, independent from the environmental temperature and independent from the pressure of gas storage 12. Thereby, a correct biopsy and a quick response of cryoinstrument 11 is also possible in case of very slim biopsy probes. This is particularly achieved without requiring the compressor to run prior to the activation or during idle phases—as a precaution, so-to-speak—and without the need to maintain any buffer vessel under pressure. In this manner, noise development and energy consumption as well as apparatus weight and volume are minimized.

In the above-mentioned embodiment compressor 20 is switched on if the pressure P1 applied at pressure sensor S1 is below the minimum pressure Pmin and it is switched off if pressure P1 is equal to or exceeds maximum pressure Pmax. If pressure P1 is inbetween at the start of activation, the flow passes compressor 20 in that the pressure of the gas from the gas storage 12 is sufficient to open the valves of compressor 20.

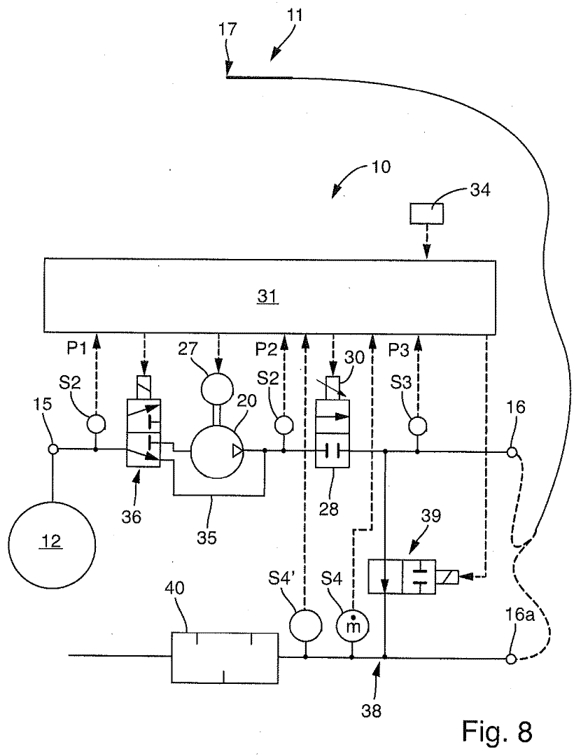

The embodiments described below (FIGS. 5 to 8), however, avoid the pressure loss occurring at these valves in that, for this mode of operation, an individual bypass channel 35 is respectively provided.

For this purpose, FIG. 5 illustrates on the basis of all reference signs introduced so far and on the basis of the associated description an embodiment in which the bypass channel 35 bypasses compressor 20. In this bypass channel 35, a valve, particularly a 2-port/2-way-valve 36 is arranged, which can be transferred by means of an actuator 37 in a closed position or open position. For this purpose, actuator 37 is connected to the control device 31. The latter is configured to stop the motor 27 and to open the 2-port/2-way-valve 36 by means of actuator 37 if and as long as the pressure P1 at pressure sensor S1 exceeds the minimum pressure Pmin.

Another variant is shown in FIG. 6 in which the bypass channel 35 is realized inside valve 28. The latter is configured as 3-port/3-way-valve for this purpose, wherein in a first valve position a complete closure of gas path 19 is present, while in a second valve position bypass channel 35 is part of the gas path. In a third valve position (illustrated in FIG. 6) compressor 20 is part of the gas path.

Apart therefrom, the above description applies on the basis of the already introduced reference signs accordingly.

A modified embodiment of the gas dosing unit 10 according to the invention is illustrated in FIG. 7. The gas dosing unit 10 is largely identical with gas dosing unit 10 according to FIG. 5. Insofar, the above description applies accordingly on the basis of the following remarks: The sensor S4 is configured as mass flow sensor that is not necessarily based on the detection of a pressure difference. Other measurement principles for flow sensors can be used. A second difference is provided in the bypass valve 36 that is here configured as 3-port/2-way-valve and is arranged between gas storage 12 and compressor 20. It forms a switch that guides the gas flow coming from the gas storage 12 either toward compressor 20 or via bypass 35 around the latter. The valve 36 is controlled by control device 31. With regard to the function of this gas dosing unit 10, reference is made to the description of the embodiment according to FIG. 5, which applies accordingly.

Another embodiment of gas dosing unit 10 is apparent from FIG. 8. The gas dosing unit 10 illustrated there is based on the gas dosing unit according to FIG. 7, the description of which applies here accordingly. Different to the above-described gas dosing units, this gas dosing unit comprises, however, a feedback channel 38 to which a feedback line 16a of instrument 11 can be connected. Between the pressurized line leading to the outlet connection 16 and the gas feedback channel 38, a vent valve 39 can be provided, which can be controlled by control device 38 similar to all other actuators. The sensor S4 can be a mass flow sensor that is now arranged in the feedback line 38 different to the above-described embodiments. At least optionally, the feedback line can also comprise an additional sensor S4′. Both sensors S4, S4′ are connected with the control device 31. In addition, a sound absorber 40 can be provided in the feedback line 38 leading to a gas exit.

Such a gas feedback channel can also be used in all of the other embodiments according to FIG. 2, 5, 6 or 7. In addition, it is reasonable to temper, particularly heat, components following compressor 20, particularly the lines and the valves 28, 39 and as necessary also valve 36 in order to avoid condensation of the compressed gas. For this purpose, a heating device can be provided that encloses the respective components. With or without tempering device, finally a gas dosing unit 10 is provided that is able to guarantee correct operation also in case of cold gas bottles, low environmental temperatures, nearly empty gas bottles as well as when using very slim and/or very long cryoprobes. Such cryoprobes can have lengths of up to and over 2.55 m and a diameter of only 1.1 down to 0.9 mm or smaller, wherein the gas dosing unit 10 according to the invention is able to overcome the increased flow resistances reliably. Such probes are particularly suitable for biopsy or also for treatment of tissue in urology, for example for biopsy, for diagnosis of suspicious tissue in the upper urinary tract and in the field of pneumology. It is suitable for miniaturized bronchoscopes with working channels that, for example, have only a clearance of 1.2 mm. Accordingly, the invention is also directed to a total arrangement consisting of instrument 11 and gas dosing unit 10 as well as the medical use thereof for biopsy in the field of urology and/or pneumology.

The gas dosing unit 10 according to the invention is configured to adjust a desired gas flow m (particularly CO₂ stream) and comprises a pressure control module 18 having a pressure reduction device 21. In case of normal room temperature, control device 31 controls the pressure reduction device 21 for adjusting a desired pressure or a desired gas stream. If the room temperature and/or the temperature of the connected gas storage 12 is lower and accordingly, the gas pressure applied at the inlet connection 15 of the gas dosing unit 10 is insufficient for the correct operation of the pressure control module, the control device activates a compressor 20 arranged in the gas path, which increases the pressure upstream the pressure control module 18 on a value appropriate for the correct operation of the pressure control module 21. In doing so, the cryoinstrument 11 can be correctly activated and used independent from the room temperature and independent from the pressure inside the pressure storage 12. Operating limitations existing otherwise are avoided.

### REFERENCE SIGNS

10 gas dosing unit
11 cryoinstrument
12 gas storage
13 connection of gas storage
14 line
15 inlet connection
16 outlet connection
16a feedback connection
17 tip
18 pressure control module
19, 19′ gas path
V1 volume of gas path between compressor 20 und valve 28
20 compressor
21 pressure reduction device
22, 23 cylinder
24, 25 connection rods
26 crankshaft
27 motor
S1 . . . S4 pressure sensors S4′
P1 . . . P4 pressures at pressure sensors S1 . . . . S4
28 valve
29 throttle
30 actuator
31 control device
32 indication device
33 input means
34 activation switch
Pmin minimum pressure
Pmax maximum pressure
Pdes desired pressure
m mass flow
35 bypass channel
36 2-port/2-way valve
37 actuator
38 gas feedback channel
39 vent valve
40 sound absorber

1. A gas dosing unit (10) for gas supply of a cryoinstrument (11), comprising:
an inlet connection (15) configured for connection to a gas storage (12);
an outlet connection (16) configured for connection to a cryoinstrument (11); and
a pressure control module (18) arranged between the inlet connection (15) and the outlet connection (16);
wherein the pressure control module (18) comprises a compressor (20) and a pressure reduction device (21) arranged downstream of the compressor (20) and a control device (31), wherein the control device (31) is configured to activate the compressor (20) if a pressure (P2) measured in front of the pressure reduction device (21) falls below a limit value (Pmin).

2. The gas dosing unit according to claim 1, wherein:
the compressor (20) is connected to be driven by a motor (27) operably connected with the control device (31), wherein the control device (31) is configured to drive the motor (27) with a variable rotational speed set by the control device (31).

3. The gas dosing unit according to claim 1, wherein a first pressure sensor (S1) is arranged between the inlet connection (15) and the compressor (20) and is operably connected with the control device (31), wherein the control device (31) is configured to monitor a pressure (P1) in a connected gas storage (12).

4. The gas dosing unit according to claim 3, wherein a second pressure sensor (S2) is arranged between the compressor (20) and the pressure reduction device (21) and is operably connected with the control device (31).

5. The gas dosing unit according to claim 4, further comprising a motor (27) for driving the compressor (20), wherein the control device (31) is configured to operate in a control operating mode in which the control device drives the motor (27) with a preset rotational speed (rpm) for a preset time phase or at least until the pressure (P2) measured between the compressor (20) and the pressure reduction device (21) has exceeded a preset minimum pressure (Pmin).

6. The gas dosing unit according to claim 4, wherein the control device (31) is configured to operate in a closed-loop control mode in which the control device (31) controls the rotational speed (rpm) of the motor (27) according to the pressure (P2) measured by the second pressure sensor (S2) in a feedback control manner.

7. The gas dosing unit according to claim 1, wherein the pressure reduction device (21) comprises a valve (28) arranged between the compressor (20) and the outlet connection (16).

8. The gas dosing unit according to claim 7, wherein the valve (28) comprises an actuator (30), wherein the valve has an open position and a closed position.

9. The gas dosing unit according to claim 8, wherein the valve (28) is configured to take intermediate positions between the open position and the closed position depending on actuator signals of the control device (31).

10. The gas dosing unit according to claim 8, wherein the control device (31) is operably connected with an activation switch (34) and is configured to stop the compressor (20) and to transition the valve (28) into the closed position if the activation switch (34) is not activated.

11. The gas dosing unit according to claim 7, wherein a third pressure sensor (S3) for detection of a third pressure (P3) and a mass flow sensor (S4) are arranged downstream of the valve (28), wherein the mass flow sensor (S4) is operably connected with the control device (31), wherein the control device (31) is configured to control the valve (28) so that a mass flow (m) at the outlet connection (16, 16a) has a predefined value.

12. The gas dosing unit according to claim 4,
wherein the control device (31) is configured to stop the compressor (20) if the pressure (P1) detected by the first pressure sensor (S1) is at least as high as a maximum pressure (Pmax); and/or
wherein the control device (31) is configured to stop the compressor (20) if the pressure (P2) measured by the second pressure sensor (S2) is at least as high as a maximum pressure (Pmax).

13. The gas dosing unit according to claim 4,
wherein the control device is configured to cause the compressor (20) to be bypassed by means of a bypass channel (35) if the pressure (P1) measured by the first pressure sensor (S1) is higher than a minimum pressure (Pmin); and
wherein the control device (31) is configured to cause the compressor (20) to be bypassed by the bypass channel (35) if the pressure (P2) detected by the second pressure sensor (S2) is higher than a minimum pressure (Pmin).

14. The gas dosing unit according to claim 1, wherein the compressor (20) comprises at least two cylinders (22, 23) with oppositely moving pistons.

15. The gas dosing unit according to claim 1, wherein the compressor (20) has a displacement volume (VK) and wherein a line (19′) a line volume (V1) is disposed between the compressor (20) and the pressure reduction device (21), wherein a ratio of the displacement volume (VK) and the line volume (V1) is higher than 1:5.

16. A method for collecting a tissue sample utilizing a cryoinstrument (11) and a gas dosing unit (10) for supplying gas to the cryoinstrument (11), the gas dosing unit comprising:
an inlet connection (15) configured for connection to a gas storage (12);
an outlet connection (16) configured for connection to a cryoinstrument (11); and
a pressure control module (18) arranged between the inlet connection (15) and the outlet connection (16);
wherein the cryoinstrument (11) comprises a flexible probe hose and has a length of at least 1.5 meters;
wherein the method comprises:
extracting a tissue sample from a bile duct of a patient utilizing the cryoinstrument (11) operably connected to the outlet connection (16) of the gas dosing unit (10).

17. The method of claim 16, wherein the cryoinstrument (11) has a length of at most 2.5 meters and an outer diameter of less than 2 millimeters.

18. A method for collecting a tissue sample utilizing a cryoinstrument (11) and a gas dosing unit (10) for supplying gas to the cryoinstrument (11), the gas dosing unit comprising:
an inlet connection (15) configured for connection to a gas storage (12);
an outlet connection (16) configured for connection to a cryoinstrument (11); and
a pressure control module (18) arranged between the inlet connection (15) and the outlet connection (16);
wherein the cryoinstrument (11) comprises a flexible probe hose and has an outer diameter of less than 2 millimeters;
wherein the method comprises:
extracting a tissue sample from a lumen of a patient utilizing the cryoinstrument (11) operably connected to the outlet connection (16) of the gas dosing unit (10).

19. The method of claim 18, wherein the cryoinstrument (11) has an outer diameter of 0.9 millimeters to 2 millimeters.

20. A system for collecting a tissue sample from a patient, comprising:
a gas dosing unit (10) for supplying gas to a cryoinstrument (11), the gas dosing unit (10) comprising:
an inlet connection (15) configured for connection to a gas storage (12);
an outlet connection (16) configured for connection to a cryoinstrument (11); and
a pressure control module (18) arranged between the inlet connection (15) and the outlet connection (16);
wherein the pressure control module (18) comprises a compressor (20) and a pressure reduction device (21) arranged downstream of the compressor (20) and a control device (31), wherein the control device (31) is configured to activate the compressor (20) if a pressure (P2) measured in front of the pressure reduction device (21) falls below a limit value (Pmin); and
a cryoinstrument (11) comprising a flexible probe hose, wherein the cryoinstrument (11) is configured to be operably connected to the outlet connection (16) of the gas dosing unit (10) and is configured for collecting a tissue sample from a patient, wherein the cryoinstrument (11) has a length of at least 1.5 meters and an outer diameter of less than 2 millimeters.

21. The system of claim 20, wherein the cryoinstrument (11) has an outer diameter of 0.9 millimeters to 1.1 millimeters.

22. The system of claim 20, wherein the cryoinstrument (11) has a length of at most 2.5 meters.
